(19) United States
(12) Patent Application Publication
(10) Pub. No.: US 2024/0238235 A1
(43) Pub. Date: Jul. 18, 2024

(54) METHODS AND COMPOSITIONS FOR TREATING PULMONARY ALVEOLAR PROTEINOSIS RELATED TO MARS MUTATIONS

(71) Applicants: INSTITUT NATIONAL DE LA SANTÉ ET DE LA RECHERCHE MÉDICALE, Paris (FR); UNIVERSITE PARIS CITE, Paris (FR); CENTRE NATIONAL DE LA RECHERCHE SCIENTIFIQUE (CNRS), Paris (FR); FONDATION IMAGINE, Paris (FR); ASSISTANCE PUBLIQUE-HÔPITAUX DE PARIS (APHP), Paris (FR)

(72) Inventors: Alice HADCHOUEL-DUVERGE, Paris (FR); Manuel SCHIFF, Paris (FR); Clément PONTOIZEAU, Paris (FR)

(21) Appl. No.: 18/561,797

(22) PCT Filed: May 25, 2022
(86) PCT No.: PCT/EP2022/064179

§ 371 Date: Nov. 17, 2023

(30) Foreign Application Priority Data
May 26, 2021 (EP) 21305689.8

(51) Int. Cl. A61K 31/198; A61K 9/00; A61P 11/00; G01N 33/68

(52) U.S. Cl. CPC A61K 31/198; A61K 9/0053; A61P 11/00; G01N 33/6884; G01N 2800/12

(57) ABSTRACT

The present invention relates to a method for treating pulmonary alveolar proteinosis related to MARS gene mutations in a subject in need thereof comprising a step of administering said subject with a therapeutically effective amount of supplementation of methionine and/or its derivatives. Pulmonary alveolar proteinosis related to mutations in the gene encoding the methionine tRNA synthetase is a severe, early-onset lung disease that also associates liver involvement, failure to thrive, and systemic inflammation. Inventors describe an infant affected by this disease who was successfully treated by oral methionine supplementation. After three months of treatment she was free of respiratory symptoms, inflammation and cholestasis resolved, and there was a catchup in growth. Her bronchoalveolar lavage fluid was free of extracellular lipoproteinaceous material. Functional assays on peripheral monocytes, initially altered, normalized. This study paves the way for similar strategies in other tRNA synthetase deficiencies.

### FIELD OF THE INVENTION

The invention is in the field of medicine, more particularly, the invention relates to methods and compositions for treating pulmonary alveolar proteinosis related to mars gene and/or protein mutations.

### BACKGROUND OF THE INVENTION

Pulmonary alveolar proteinosis (PAP) is a rare chronic interstitial lung disease characterized by alveolar accumulation of lipoproteinaceous material derived from surfactant.¹ The diagnosis is suggested by chest computed tomography (CT), showing a “crazy paving” pattern and alveolar consolidations,² and is confirmed by periodic acid-Schiff (PAS) staining of bronchoalveolar lavage fluid (BALF).³ We previously described 34 children with a specific type of PAP prevalent on La Réunion Island, characterized by an early onset, associated liver involvement, systemic inflammation, poor prognosis, and frequent progression to lung fibrosis, despite whole-lung lavages (WLL).⁴ Bi-allelic mutations in MARS were subsequently identified as disease causing.⁵ MARS encodes the cytosolic methionine tRNA synthetase (MetRS), which belongs to the class 1 family of aminoacyl-tRNA synthetases (ARS). These enzymes play a critical role in protein biosynthesis by charging tRNAs with their cognate amino acids, leading to the formation of aminoacyl-tRNA. MetRS also has an editing and proofreading function to ensure translational fidelity⁶ and is also a component of a cytosolic multiprotein complex (the MSC; multiaminoacyl-tRNA synthetase complex), with multiple roles in the immune response, inflammation, tumorigenesis, angiogenesis, and neuronal homeostasis.^{7,8} The bi-allelic Ala393Thr/Ser567Leu mutations found in Réunion patients are located in the catalytic domain of MetRS and severely impair growth and enzyme activity in yeast that are restored by methionine supplementation in the culture medium.⁵ Enzymatic preparations purified from transfected E. coli have confirmed the significant impact of the mutations on the rate of the aminoacylation reaction (reduction of the kcat by 5 to 6-fold relative to wildtype), especially for methionine affinity, as shown by a significant increase in the Km for methionine in all mutants.⁹ Co-immuno-precipitation experiments in Hela cells showed that these mutations do not alter the ability of MetRS to associate with other components of the MSC.⁹

Currently, there is not any therapeutics solution to treat pulmonary alveolar proteinosis (PAP) related to MARS gene mutations.

### SUMMARY OF THE INVENTION

The invention relates to a method for treating pulmonary alveolar proteinosis related to MARS gene and/or protein mutations in a subject in need thereof comprising a step of administering said subject with a therapeutically effective amount of supplementation of methionine and/or its derivatives. In particular, the invention is defined by claims.

### DETAILED DESCRIPTION OF THE INVENTION

Pulmonary alveolar proteinosis related to mutations in the gene encoding the methionine tRNA synthetase is a severe, early-onset lung disease that also associates liver involvement, failure to thrive, and systemic inflammation. Inventors describe an infant affected by this disease who was successfully treated by oral methionine supplementation. After three months of treatment she was free of respiratory symptoms, inflammation and cholestasis resolved, and there was a catchup in growth. Her bronchoalveolar lavage fluid was free of extracellular lipoproteinaceous material. Functional assays on peripheral monocytes, initially altered, normalized. This study paves the way for similar strategies in other tRNA synthetase deficiencies.

Accordingly, in a first aspect, the invention relates to a method for treating pulmonary alveolar proteinosis related to MARS gene and/or protein mutations in a subject in need thereof comprising a step of administering said subject with a therapeutically effective amount of methionine and/or its derivatives.

In a particular embodiment, the invention relates to a method for treating pulmonary alveolar proteinosis related to MARS gene and/or protein mutations in a subject in need thereof comprising a step of administering said subject with a therapeutically effective amount of supplementation of methionine.

In a particular embodiment, the invention relates to the methionine or its derivatives for use in the treatment of pulmonary alveolar proteinosis related to MARS gene and/or protein mutations in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pulmonary alveolar proteinosis” refers to a rare chronic interstitial lung disease characterized by alveolar accumulation of lipoproteinaceous material derived from surfactant. In the context of the invention, the PAP is related to MARS gene and/protein mutations.

As used herein, the term “MARS” refers to Methionyl-tRNA synthetase, is an enzyme cytoplasmic that in humans is encoded by the MARS gene. These enzymes play a critical role in protein biosynthesis by charging tRNAs with their cognate amino acids. The encoded protein is a component of the multi-tRNA synthetase complex and catalyzes the ligation of methionine to tRNA molecules. The naturally occurring human MARS has a nucleotide sequence as shown in Genbank Accession number NM_004990 and the naturally occurring human MARS protein has an amino acid sequence as shown in Genbank Accession number NP_004981. The naturally occurring murine MARS has a nucleotide sequence as shown in Genbank Accession numbers NM_001003913 and NM_001171582; and the naturally occurring murine MARS protein has an amino acid sequence as shown in Genbank Accession numbers NP 001003913 and NP_001165053.

As used herein, the term “MARS gene and/or protein mutations” refers to any mutations in MARS gene and/or protein. As used herein, the term “mutation” has its general meaning in the art and refers to any detectable change in genetic material, e.g. DNA, RNA, cDNA, or in an amino acid sequence encoded by such a genetic material. This includes gene mutations, in which the structure (e.g. DNA sequence) of a gene is altered any gene as well as protein mutations, in which the amino-acid structure of the protein is altered. Generally a mutation is identified in a subject by comparing the sequence of a nucleic acid or of a polypeptide expressed by said subject with the corresponding nucleic acid or polypeptide expressed in a control population. Typically, mutations are accessible in the Single Nucleotide Polymorphism Database (dbSNP), which is a free public archive for genetic variation within and across different species developed and hosted by the National Center for Biotechnology Information (NCBI) in collaboration with the National Human Genome Research Institute (NHGRI).

Typically, such mutation refers to at least one nucleotide and/or amino acid substitution(s), deletion(s) and/or insertion(s) in the MARS gene and/or protein sequence. In the context of the invention, the MARS gene and/or protein mutation refers to a double mutation Ala393Thr/Ser567Leu in MARS. Other MARS mutations were reported in the literature (Abuduxikuer PMID: 30271085, Sun PMID: 28148924, Van Meel PMID: 24103465, Rips PMID: 29655802, Alzaid PMID: 30723866) and are also located in the catalytic domain. Therefore, those mutations and all other mutations that may be identified in the future and that are located in the catalytic domain, mays benefit from this therapeutic intervention.

As used herein, the term “subject” refers to any mammals, such as a rodent, a feline, a canine, and a primate. In a particular embodiment, the subject is human. In a particular embodiment, the subject is an adult, child or baby. Particularly, in the present invention, the subject has or is susceptible to have pulmonary alveolar proteinosis. In a particular embodiment, the subject has or is susceptible to have pulmonary alveolar proteinosis related to MARS gene and/or protein mutations.

As used herein, the term “methionine” also known as Met or M is an essential amino acid in humans. As the substrate for other amino acids such as cysteine and taurine, versatile compounds such as SAM-e, and the important antioxidant glutathione, methionine plays a critical role in the metabolism and health of many species, including humans. It is encoded by the codon AUG. Methionine is well-known in the art and has the following chemical formula C₅H₁₁NO₂S and the CAS number: 59-51-8. Methionine has two isoforms: L-isomer having CAS number 63-68-3 and D-isomer having CAS number 348-67-4.

As used herein, the term “derivatives” refers to any derivative of methionine resulting from reaction at an amino group, carboxy group, side-chain functional group or from the replacement of any hydrogen by a heteroatom.

As used herein, the term “supplementation” refers to pills, drinks or foods containing substances that people usually get from food that are given to patients who do not weigh enough or who are not able to take in enough of these substances in the food. Most essential amino acids supplied by animal diets are derived from naturally occurring plant or animal proteins. It is known, however, to supplement normal diets with various free amino acids such as lysine from lysine HCl, DL-methionine, L-tryptophan, L-isoleucine and L-threonine. The theory of such supplementation is that the free amino acids are thought to be absorbed by an animal more readily than more complex peptides and proteins.

In a second aspect, the invention relates to a method for adjusting the methionine supplementation dosage comprising the following steps: i) measuring the level of methioninemia in a biological sample obtained from a subject suffering from pulmonary alveolar proteinosis related to MARS gene and/or protein mutations; ii) comparing said level with its predetermined reference value; and concluding that there is a need to:
iii) increase methionine supplementation dosage when the level of methioninemia is inferior to the said predetermined reference value;
iv) continue the same methionine supplementation dosage at day 0 (day 0) when the level of methioninemia is same to the said predetermined reference value; or
v) reduce the methionine supplementation dosage when the level of methioninemia is superior to the said predetermined reference value.

As used herein, the term “adjusting” refers to changes that can be performed with methionine and/or its derivatives supplementation. Typically, the physician can continue, reduce or increase the doses of the methionine and/or its derivatives supplementation by measuring the level of methioninemia.

As used herein, the term “methioninemia” refers to the level of amino acid methionine in the blood. The level of methioninemia in a healthy subject should be measured on an empty stomach and should be less than 45 μM. Typically, in the subject affected by MARS mutations, the level of methioninemia to reach efficacy should be in the range of 45-500 μM (>45 μM at residual dosage and <500 μM at peak dosage 1 hour after the dose has been taken). The level of methioninemia is measured with the following methods. Proteinogenic amino acids (including methionine), citrulline, ornithine and free homocystine in plasma are measured by liquid chromatography coupled to tandem mass spectrometry (UPLC-MS/MS). For accurate quantification, stable isotope internal standards of the same structure for each metabolite (Eurisotop, Saint Aubin, France) are added to samples before protein precipitation. Internal standards are labeled on all carbons and nitrogen(s) with following exceptions: glutamine 2,3,3,4,4-D5, asparagine ¹³C₄, tryptophan Indole-D5, citrulline ¹³CD₄ and homocysteine 3,3,3′,3′,4,4,4′,4′-D8. Samples are first derivatized using the AccQ Tag™ Ultra (Waters Corporation, Milford, MA, USA) according to manufacturer recommendations. Amino acid separation is performed with an Acquity™ UPLC system using a CORTECS™ UPLC C₁₈ column (1.6 μm, 2.1×150 mm) coupled to microTQS™ tandem mass spectrometer (Waters Corporation, Milford, MA, USA). The run time for quantification of all amino acids is 12 min. A second method for methionine and free homocystine fast quantification was derived from the previous one through optimization of chromatographic conditions, resulting in a run time of 3 min.

As used herein, the term “biological sample” refers to any sample obtained from a subject, such as a serum sample, a plasma sample, a urine sample, a blood sample, a lymph sample, tumor sample or a tissue biopsy. In a particular embodiment, biological sample for the determination of methioninemia level includes samples such as a blood sample, a lymph sample, or a biopsy. In the context of the invention, the biological sample is a blood sample. In another embodiment, the biological sample is a plasma sample.

As used herein, the term “subject” denotes a mammal, such as a rodent, a feline, a canine, and a primate. Particularly, the subject according to the invention is a human. In a particular embodiment, the subject is an adult, child or baby. More particularly, the subject according to the invention has or is susceptible to have pulmonary alveolar proteinosis related to MARS gene and/or protein mutations.

Typically, the predetermined reference value is a threshold value or a cut-off value, which can be determined experimentally, empirically, or theoretically. A threshold value can also be arbitrarily selected based upon the existing experimental and/or clinical conditions, as would be recognized by a person of ordinary skilled in the art. For example, retrospective measurement of the level methioninemia in properly banked historical plasma samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of methioninemia in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In the context of the invention, the predetermined reference value refers to the methioninemia measured in a healthy person. Typically the predetermined reference value of said methioninemia is below 45 μM. In a particular embodiment, the predetermined reference value in a subject affected by MARS mutations is in the range of 45-500 μM. In order to reach clinical efficacy, levels of methioninemia in affected subjects should be over the reference value but below toxic values. In a particular embodiment, the Toxic values are determined by available data in patients suffering from congenital hypermethioninemia (methionine adenosyl transferase I/III deficiency and cystathionine beta-synthase deficiency), with a threshold of 800 μM.

As used herein the terms “administering” or “administration” refer to the act of injecting or otherwise physically delivering a substance as it exists outside the body (e.g. methionine supplementation or increase of methionine supplementation) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In the context of the invention, the methionine is administered orally. The method according to the invention, wherein the methionine is formulated for an oral administration. In a particular embodiment, the methionine is formulated as tablets, gel capsules, powders, granules and oral suspensions or solutions, sublingual, oral suspension and buccal administration forms.

By a “therapeutically effective amount” is meant a sufficient amount of methionine for use in a method for the treatment of pulmonary alveolar proteinosis related to MARS gene and/or protein mutations at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient (methionine) for the symptomatic adjustment of the dosage to the subject to be treated. A medicament typically contains from about 0.01 mg to about 500 mg of the active ingredient (e.g. methionine), typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. In a further embodiment, the therapeutically effective amount of methionine supplementation is in the range of 60 and 100 mg/kg/day. In a further embodiment, the therapeutically effective amount of methionine supplementation is selected from the group consisting of but not limited to 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/kg/day. In a particular embodiment, the methionine supplementation is administered 4 times every 6 hours.

In the context of the invention, the therapeutically effective amount of methionine and/or its derivatives as described above is sufficient to reduce and/or control respiratory symptoms, resolve inflammation and cholestasis, increase the catch-up in growth and to free bronchoalveolar lavage fluid of extracellular lipoproteinaceous material.

In a third aspect, the invention relates to a pharmaceutical composition comprising methionine and/or its derivatives. The pharmaceutical composition according to the invention, can be used for the treatment of pulmonary alveolar proteinosis related to MARS gene and/or protein mutations in a subject in need thereof.

In a particular embodiment, the pharmaceutical composition according to the invention comprising methionine and/or its derivatives as a combined preparation for simultaneous, separate or sequential use in the treatment of pulmonary alveolar proteinosis related to MARS gene and/or protein mutations.

More particularly, the pharmaceutical composition according to the invention is suitable to reduce and control respiratory symptoms, resolve inflammation and cholestasis, increase the catch-up in growth and to free bronchoalveolar lavage fluid of extracellular lipoproteinaceous material.

The pharmaceutical composition according to the invention for use in the treatment of pulmonary alveolar proteinosis related to MARS gene and/or protein mutations in a subject in need thereof.

The methionine and/or its derivatives as defined above and the pharmaceutical combination according to the invention, as described above may be combined with pharmaceutically acceptable excipients, and optionally sustained-release matrices, such as biodegradable polymers, to form pharmaceutical compositions. In a further embodiment, methionine and/or its derivatives as defined above is combined with mannitol as a pharmaceutically acceptable excipient.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a excipient, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In some embodiments, the pharmaceutical formulation can be suitable orally, subcutaneously, intradermally, ocularly or topically administration. In a particular embodiment, the pharmaceutical formulation is suitable for oral administration.

In a fourth aspect, the invention relates to a kit for performing the method according to the invention, wherein said kit comprises (i) means for measuring the level of methioninemia in a biological sample from a subject suffering from pulmonary alveolar proteinosis related to MARS gene mutations and (ii) instructions to adjust the methionine and/or its derivatives supplementation dosage.

Typically such kit allows to compare the level of methioninemia with its predetermined reference value; and conclude that there is a need to:
I) increase methionine and/or its derivatives supplementation dosage when the level of methioninemia is inferior to the said predetermined reference value;
ii) continue the same methionine and/or its derivatives supplementation dosage at day 0 (day 0) when the level of methioninemia is same to the said predetermined reference value; or
ii) reduce the methionine and/or its derivatives supplementation dosage when the level of methioninemia is superior to the said predetermined reference value.

The invention will be further illustrated by the following figures and examples. However, these examples and figures should not be interpreted in any way as limiting the scope of the present invention.

### FIGURES

FIG. 1. Pharmacokinetic data of residual (solid lines) and peak (dotted lines) plasma methionine values. The peak was determined during a kinetic study to be 1 h after taking the medication. A and B: Complete kinetic study measuring the residual and peak concentrations at each intake over 24 h. Data are shown for Patient 1 after one year of treatment (A) with a methionine dose of 110 mg/kg/day and for Patient 3 on day 3 of the treatment (B) with a methionine dose of 80 mg/kg/day. C and D: Residual and peak plasma values on three different days under the same dosage. Data are shown for Patient 2 after 10 (M10), 11 (M11), and 13 (M13) months of treatment with a methionine dose of 80 mg/kg/day and for Patient 4 on three different days (D5, D10, and D11) during the same month (M2) of treatment with a methionine dose of 90 mg/kg/day. For all the patients, giving treatment every 6 hours allowed getting reproducible residual and peak values during 24-hour periods and also across days and months of treatment.

FIG. 2. Cellular analyses for Patients 1 and 3. A: Analysis of MetRS protein expression for Patient 2. The MetRS protein was normally expressed in PBMCs relative to those of a control individual. B: GM-CSF priming of ROS production by peripheral monocytes before (MO) and after three months of treatment (M3) for Patient 1 (P1) and Patient 3 (P3). Priming of ROS production by peripheral monocytes stimulated by GM-CSF and fMLP was measured in patients and controls and the stimulation index expressed as the percentage of the control values. The control value was thus considered to be 100%. For both patients, before treatment, the stimulation index relative to control for both patients before treatment was low: 46% for P1 and 58% for P3. After three months of treatment, the stimulation index relative to control normalized for P1 (109%) and improved for P3 (73%).

TABLE 1
Patient characteristics at inclusion.

| | P1 | P2 | P3 | P4 |
|---|---|---|---|---|
| Ethnic origin | Réunion | Mayotte | Réunion | Comoros |
| Gender | Girl | Girl | Girl | Boy |
| Age at diagnosis | 4 | 5 | 3 | 3 |
| (months) | | | | |
| Age at inclusion | 6 | 35 | 6 | 21 |
| (months) | | | | |
| Respiratory status | | | | |
| Oxygen | Yes | No | Yes | Yes |
| dependency | | | | |
| Ventilatory | No | No | No | Yes |
| support | | | | |
| Growth and | | | | |
| nutrition | | | | |
| Weight (SD) | 4.2 (−3.4) | 14.9 (+1) | 4.1 (−3.7) | 8.4 (−2.7) |
| Regimen | Enteral (65%) + | Complete | Enteral (70%) + | Complete |
| | Oral (35%) | enteral | Oral (30%) | parenteral |
| | | nutrition | | nutrition |
| Vomiting | Yes | Yes | Yes | Yes |
| Liver status | | | | |
| Enlarged liver | Yes | Yes | Yes | Yes |
| Elevated | Yes | No | Yes | No |
| AST/ALT | | | | |
| Elevated GGT | Yes | No | Yes | Yes |
| Hematological | | | | |
| status | | | | |
| Anemia | Yes | No | Yes | Yes |
| High neutrophils' | Yes | No | Yes | Yes |
| count | | | | |
| Thrombocytosis | Yes | No | No | No |
| Inflammation* | Yes | Yes | Yes | Yes |
| Daily methionine | 231 | 650 | 256 | 297 |
| intake | | | | |
| at baseline (mg) | | | | |
| Fasting | 18 | 33 | 13 | 23 |
| methionine | | | | |
| plasma | | | | |
| concentration | | | | |
| (μM) | | | | |

P: patient.
*Inflammation was diagnosed by the presence of a high level of CRP, a high sedimentation rate, or both.

TABLE 2
Disease course under methionine supplementation. Patient 1
and 2. LFU: last follow-up. D 60: day 60 = end of the trial

| | P1 | | | P2 | | |
|---|---|---|---|---|---|---|
| | Inclusion | | LFU 19 | Inclusion | | LFU 4 |
| | 6 months | D 60 | months | 3 years | D 60 | years |
| Clinical features | | | | | | |
| Weight (kg) (SD) | 4.2 (−3.4) | 4.9 (−3.3) | 10.2 (0) | 14.9 (+1) | 15 (+1) | 18.5 (+2) |
| Respiratory rate | 70 | 32 | 32 | 28 | 24 | 24 |
| (cycles/min) | | | | | | |
| Oxygen dependency | 1 L/min | No | No | No | No | No |
| Nutritional regimen | 65% E | 100% O | 100% O | 100% E | 80% E | 40% E |
| | 35% O | | | | 20% O | 60% O |
| Biological features | | | | | | |
| Hemoglobin (g/dL) | 9.1 | 10.4 | 11.7 | 11 | 11.3 | 11.4 |
| Leucocytes (/mm³) | 31,700 | 5,100 | 6,900 | 6,400 | 9,200 | 7,400 |
| Neutrophils (/mm³) | 18,100 | 1,900 | 1,400 | 1,700 | 4,500 | 3,000 |
| Platelets (/mm³) | 668,000 | 218,000 | 339,000 | 157,000 | 235,000 | 204,000 |
| Sedimentation rate | 56 | 46 | 25 | 11 | 7 | NA |
| (mm) | | | | | | |
| CRP (mg/L) | 53.8 | 8.3 | 2.8 | 0.5 | 0.5 | <0.5 |
| IgG (g/L) | 21.52 | 10.94 | 8.03 | 9.23 | 10.89 | 7.39 |
| Albumin (g/L) | 23.2 | 38.4 | 42.3 | 38 | 44.2 | 39.7 |
| Prothrombin rate (%) | 54 | 71 | 86 | 91 | 85 | 92 |
| AST (IU/L) | 81 | 44 | 52 | 45 | 28 | NA |
| ALT (IU/L) | 23 | 16 | 25 | 28 | 22 | 16 |
| GGT (IU/L) | 64 | 44 | 13 | 16 | 17 | 10 |
| PAL (IU/L) | 296 | 447 | 475 | 237 | 216 | 213 |
| T bilirubin (μM) | 47 | 4 | 4 | 4 | 4 | 4 |
| C bilirubin (μM) | 36 | 2 | 0 | 0 | 0 | 0 |

TABLE 3
Disease course under methionine supplementation. Patient 3
and 4. LFU: last follow-up. D 60: day 60 = end of the trial

| | P3 | | | P4 | | |
|---|---|---|---|---|---|---|
| | Inclusion | | LFU 11 | Inclusion | | LFU 25 |
| | 6 months | D 60 | months | 21 months | D 60 | months |
| Clinical features | | | | | | |
| Weight (kg) (SD) | 4 (−3.7) | 5.3 (−3) | 6.4 (−2.5) | 8.4 (−2.7) | 10 (−1.5) | 11.5 (−0.7) |
| Respiratory rate | 60 | 55 | 40 | 65 | 65 | 42 |
| (cycles/min) | | | | | | |
| Oxygen | 0.5 L/min | No | No | 2-4 L/ | D: 0.5 | 0.5 L/min |
| dependency | | | | min + | L/min | |
| | | | | c-NIV | N: 0.8 | |
| | | | | | L/min | |
| Nutritional | 70% E | 35% E | 100% O | 100% P | 80% P | 100% E |
| regimen | 30% O | 65% O | | | 20% E | |
| Biological features | | | | | | |
| Hemoglobin | 8.7 | 10.1 | 9.6 | 9.2 | 9.3 | 11.5 |
| (g/dL) | | | | | | |
| Leucocytes | 20,900 | 13,500 | 13,100 | 18,820 | 8,200 | 7,700 |
| (/mm³) | | | | | | |
| Neutrophils | 11,200 | 3,100 | 3,000 | 14,490 | 3,100 | 2,200 |
| (/mm³) | | | | | | |
| Platelets (/mm³) | 441,000 | 565,000 | 450,000 | 189,000 | 183,000 | 165,000 |
| Sedimentation | 87 | 43 | 48 | 34 | 27 | 41 |
| rate (mm) | | | | | | |
| CRP (mg/L) | 15.8 | 0.8 | 1.4 | 71.4 | 1.1 | 1.1 |
| IgG (g/L) | 18.03 | 11.3 | 13.17 | 15.07 | 11.32 | 13.83 |
| Albumin (g/L) | 28.7 | 43 | 42.8 | 6 | 26.5 | 30 |
| Prothrombin rate | 72 | 85 | 82 | 81 | 71 | 80 |
| (%) | | | | | | |
| AST (IU/L) | 101 | 59 | 48 | 50 | 108 | 98 |
| ALT (IU/L) | 65 | 26 | 22 | 7 | 44 | 35 |
| GGT (IU/L) | 406 | 94 | 16 | 198 | 214 | 61 |
| PAL (IU/L) | 247 | 223 | 175 | 296 | 372 | 382 |
| T bilirubin (μM) | 4 | 2 | 2 | 30 | 13 | 11 |
| C bilirubin (μM) | 4 | 0 | 0 | 13 | 0 | 0 |

### EXAMPLE

### Material & Methods

### Study Protocol

The MetPAP study was registered at clinicaltrials.gov (NCT03887169). Its main objective was to determine the safety and tolerance of prolonged daily oral supplementation of methionine in patients presenting pulmonary alveolar proteinosis due to the double mutation Ala393Thr/Ser567Leu in MARS. The secondary objectives were to determine the efficacy of such treatment. The patient was given methionine orally or enterally for two months. L-methionine was given every 6 h, starting at 80 mg/kg/day and progressively increased until obtaining plasma concentrations between 45 and 500 μM at residual and peak dosages (1 h after intake).

The inclusion criteria were: a child affected by PAP related to the double mutation Ala393Thr/Ser567Leu in MARS, patient requiring WLL, possibility to administrate methionine orally or by the enteral route (nasogastric feeding tube or gastrostomy), written informed consent signed by the parents. The exclusion criteria were: patient presenting with PAP related to other MARS mutations, patient presenting with PAP related to another cause, systemic arterial hypertension requiring pharmacological treatment, cardiac failure, known hypersensitivity or allergy to methionine and/or concomitant treatments potentially used in the study (i.e. vitamins B6, B9, and B12), prior high plasma concentration of methionine (>2 standard deviations (SD)), parental refusal.

The frequency of medication was based on the known half-life of the moleculel and the peak was determined by performing kinetic measurements on the patients during the first day of supplementation. The initial dosage was determined based on the usual mean methionine intake in alimentation infants and for children (available at https://www.anses.fr/fr/system/files/NUT-Ra-Proteines.pdf), with the initial aim to double the methionine intake. The targeted plasma concentrations were defined according to available published data on normal methionine concentrations in children and on congenital disorders leading to hypermethioninemia and its potential toxicity. The normal fasting concentration should not exceed 45 μM.² Congenital hypermethioninemia is described in patients with methionine adenosyltransferase I/III (MAT I/III) or cystathionine beta-synthase deficiency. The consequences of high blood levels of methionine in these patients are liver dysfunction and central nervous system (CNS) abnormalities, especially with a risk of cerebral edema. In a large series of patients with MAT I/III deficiency, CNS abnormalities were observed in patients with mean plasma methionine values >800 μM, whereas patients with mean plasma methionine values <800 μM usually do not have such abnormalities.³ We decided to target methionine plasma levels between 50 and 500 μM to obtain levels above the normal rang but below the toxic range.

Potential adverse effects included the neurological and liver side effects described above but also adverse events described in subjects receiving a loading dose of methionine in research that studied the relationship between homocysteine, which is derived from methionine metabolism, and cardiovascular disease. These effects were mild and always transient, with a moderate increase or decrease in arterial blood pressure, nausea and vomiting, dizziness, and polyuria.⁴ Homocysteine plasma levels were also monitored and supplementation with vitamins B6, B9, and B12 was initiated when levels exceeded the normal range (i.e. 10 μM) to favor the transformation of homocysteine back to methionine.⁵

Efficacy of the treatment was evaluated based on the respiratory, hepatic, inflammatory, and growth status. Respiratory assessment included regular clinical evaluation of the respiratory rate, signs of chest retraction and the need for oxygen, chest CT scan at inclusion and at the end of treatment, pathological aspects of broncho-alveolar lavage fluid, and the possibility to space out the WLLs. Liver status was assessed by clinical examination, liver ultrasound scan (US), and liver function tests (AST, ALT, GGT, PAL, bilirubinemia). Growth and nutritional status were assessed by monitoring growth charts and albuminemia. Systemic inflammation was assessed by measuring CRP, the erythrocyte sedimentation rate, and IgG levels.

### Comparison to the Historical Controls (Hc)

We compared the course of treated patients to that of patients reported by Enaud et al. 2 as well as seven patients diagnosed since this publication. Data collection and analysis for these patients were approved by the Institutional Review Board of the French Respiratory Society (CEPRO2013-019) and the CPP Île de France II (CPP2013-12-03 SC).

### Western Blot

Neutrophils were isolated from blood of the patient and a control as described previously.⁶ After hypotonic lysis of erythrocytes, the neutrophil pellets were collected and washed in PBS. Neutrophils (107 cells in 500 μl HBSS) were then incubated with proteinase inhibitor DFP (2.5 mM), followed by lysis with 125 μl concentrated modified Laemmli sample buffer (5×) containing 50 μg/mL pepstatin, 50 μg/mL leupeptin, 25 mM NaF, 12.5 mM Na3VO4, 12.5 mM EDTA, 12.5 mM EGTA, 6.25 mM p-NPP, and 50 μg/mL aprotinin.⁷ Samples were denatured in boiling water (100° C.) for 3 min and stored at −80° C. until use. Samples were thawed and sonicated for 10 s before use and then subjected to classical 10% SDS-PAGE.⁷ The separated proteins were transferred to nitrocellulose membranes. The membranes were blocked with 5% non-fat dry milk in a mixture of tris-buffered saline and Tween-20. The membranes were incubated overnight at 4° C. in a solution containing a specific anti-MARS antibody (Abnova H00004141-B01P), followed by incubation in secondary antibodies (Santa Cruz, Heidelberg, Germany). Blots were visualized using ECL Western blotting reagents (Amersham Pharmacia).

### Priming of Reactive Oxygen Species (Ros) Production by Peripheral Phagocytes/Monocytes

Peripheral monocyte and phagocyte functions were assessed by quantifying ROS production. Whole blood collected from lithium heparinized tubes (500 μl) was incubated for 15 min at 37° C. with dihydrorhodamine 123(DHE) (Sigma-Aldrich). Samples were then treated for 1 h at 37° C. with GM-CSF (10 ng/ml; R&D Systems), followed by stimulation for 5 min with fMLF (10-5M; Sigma-Aldrich). The reaction was stopped by adding 1 ml ice-cold lysis solution (BD Biosciences) and incubating for 5 min on ice. Samples were then washed with PBS (Sigma-Aldrich) and the pellets resuspended in 300 μl FacsFlow solution (BD Biosciences). Samples were analyzed by flow cytometry on a FACSCanto II (BD Biosciences). Phagocytes (neutrophils and monocytes) were selected on the FSC-SSC dot plot. Events (50,000) were recorded at a constant PMT voltage. Results are expressed as the index of stimulation (MIF DHE GM-CSF+fMLP/DHE GM-CSF alone).

### Statistical Analyses

Several parameters were compared between treated patients and HC. We computed the difference between the values for each continuous variable at inclusion and at last follow-up for each treated patient, as well as between diagnosis and six months to one year from diagnosis for the HC. Differences were compared between groups using Mann-Whitney tests. For each categorical variable (i.e., weaning from oxygen and enteral nutrition), we compared the proportion of patients who were weaned from such support at the second assessment between groups using Fisher's exact test. A p-value<0.05 was considered statistically significant.

### Results

### Patients' Phenotypes

The patients' characteristics before treatment are presented in Table 1, 2 and 3. Patients (P) 1 and 3 were included in the trial soon after the diagnosis at six months of age. They had not yet received any treatment nor undergone WLL. P2 had already undergone 25 WLL. She received monthly IV steroid pulses and daily oral steroids from the age of 11 months. As she had become steroid-dependent, she was started on mycophenolate mofetil (MMF) at the age of 21 months, which allowed tapering then stopping the steroids at the age of 25 months, and spacing the WLL every six months. She was the first patient treated with MMF. She still showed feeding difficulties, refusing oral feeding and requiring total enteral nutrition using a gastrostomy. P4 had already undergone 19 WLL and received monthly IV steroid pulses. Despite such care, he required continuous non-invasive ventilation (NIV), supplemental oxygen and total parenteral nutrition. The repeated occurrence of severe sepsis contraindicated MMF. Regarding inflammatory parameters, in Patients 1 to 3, inflammation was persistent without any concurrent infection. In Patient 4, sepsis occurred several times but inflammation was persistent outside infectious periods. Fasting methionine plasma level before inclusion was normal in Patients 1, 2 and 4, and low in Patient 3 (Tables 2 and 3).

### Methionine Administration

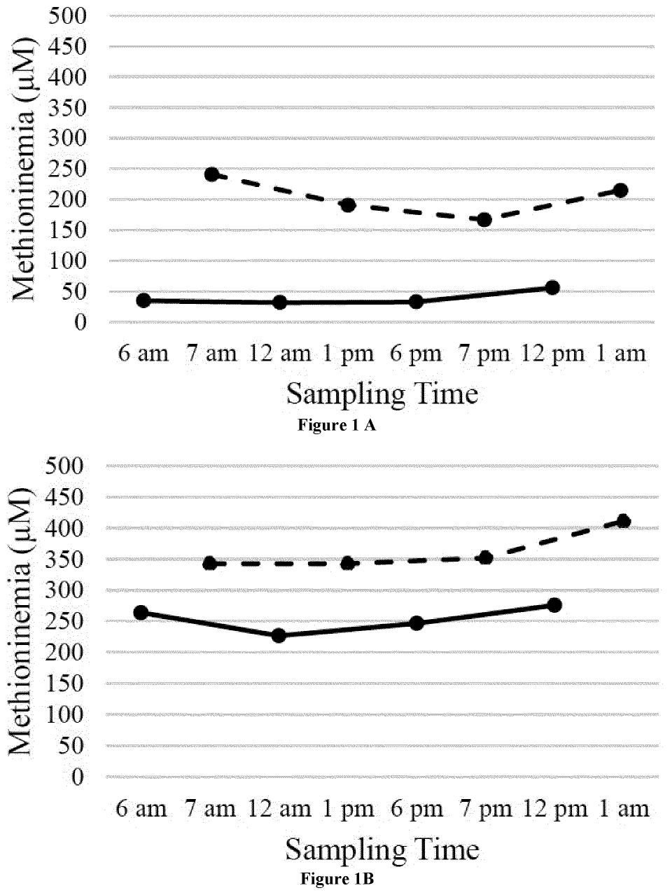

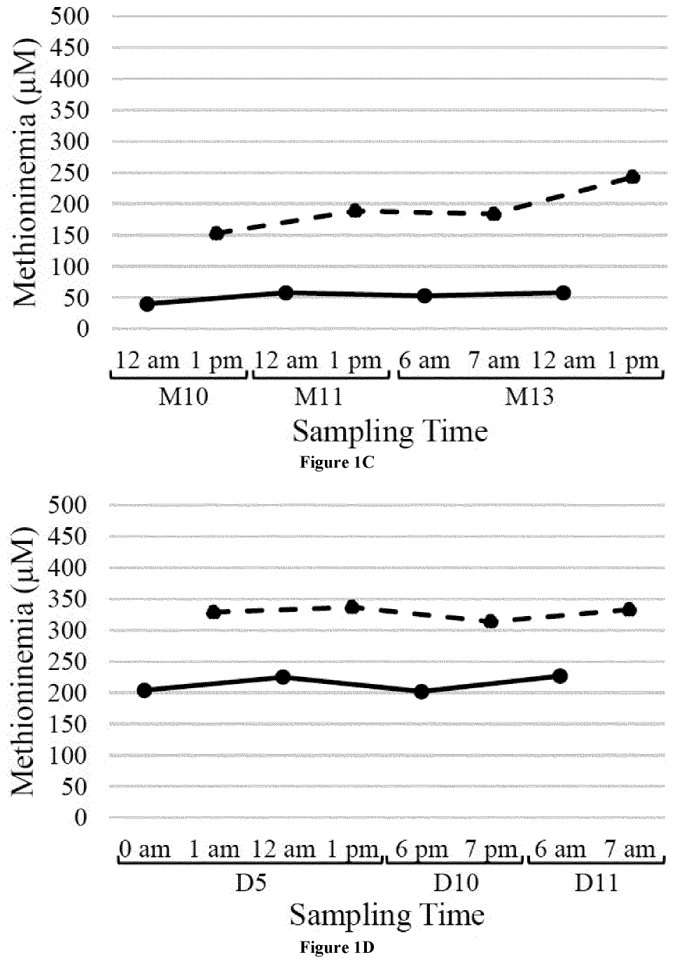

Twenty-four-hour kinetic studies were performed on days (D) 1 and 3 of treatment and at least one time later before D60. Plasma values were then regularly monitored at residual and peak states. Treatment every 6 h led to reproducible residual and peak values for 24-h periods throughout days and months of treatment (FIG. 1A, B, C, D). At the last follow-up, doses were 120 mg/kg/day for P1, 80 mg/kg/day for P2, 110 mg/kg/day for P3, and 80 mg/kg/day for P4.

### Safety of the Treatment

Methionine supplementation was well tolerated during the protocol and after. P3 presented initially mild elevated transaminases (Tables 1, 2 and 3), which normalized on D5 of treatment. On D21 she presented a new episode of elevated transaminases (>3N), which led to a reduction in methionine doses, as planned in the protocol. Indeed, as liver dysfunction has been described in congenital hypermethioninemia, the protocol provided for a reduction in the dose in the event of elevated transaminases that exceeded three times the normal value of AST and/or ALT, until resolution. Nevertheless, (i) the protocol did not anticipate analyzing the course of AST and ALT values according to methionine plasma level and (ii) in a review by Chien et al. of patients with MAT III deficiency/hypermethioninemia, none of the 30 patients with available data showed elevated AST nor ALT during their course and I. For P3, we observed an increase in AST and ALT at a time in which the plasma methionine concentration decreased. This decrease in methioninemia occurred from D7 to reach TO values below the minimum target range of 45 μM at D21 (42 μM) and is probably explained by the rapid weight gain of the child (+500 g between DI and D21 of treatment, i.e., a 12.5% increase in the patient's weight). At the same time, we observed a progressive increase in transaminases from D15, which continued, with a maximum at D21, when the methioninemia at TO was within the normal values for age (42 μM). As liver failure with elevated transaminases is itself one of the features of MARS related PAP, we hypothesized that the observed decrease in methioninemia was actually the cause of the increase in AST and ALT. A complementary analysis of the literature found data supporting this hypothesis. In rats, mice, and chickens, methionine restriction in the diet of these animals induces steatohepatitis and is a classic NASH (non-alcoholic steatohepatitis) model. We thus concluded that the occurrence of elevated transaminases at the same time that methionine plasma levels decreased in P3 was not attributable to a toxic effect of methionine supplementation. Furthermore, the decrease in methionine doses rapidly led to a deterioration of the patient's general condition with, in particular, a reappearance of vomiting and food refusal. To reintroduce the treatment at therapeutic dosages without creating a major deviation in the protocol, we decided to remove P3 from the trial. Treatment was then reintroduced at the initial dosage and resulted in an increase in methionine plasma levels, along with a rapid improvement in AST and ALT values and the general condition and the resolution of vomiting, resumption of oral feeding, and weight gain. No recurrence of elevated transaminases has occurred since. Plasma homocysteine never reached the threshold of 30 μM for any of the patients.

### Disease Course and Efficacy of Methionine Supplementation

### Patient 1

At inclusion, P1 had severe growth failure, required continuous supplemental oxygen, enteral nutrition and experienced chronic vomiting. Laboratory parameters showed anemia, cholestasis, mild elevated AST, hypoalbuminemia, inflammation and high IgG level (Table 1). Ultrasound showed hepatomegaly with hyperechoic parenchyma. Chest CT showed symmetrical ground-glass opacities, intralobular lines, and thickened interlobular septa (data not shown). Bronchoalveolar lavage fluid (BALF) was macroscopically opalescent and pathological examination was typical of PAP (data not shown). She underwent seven therapeutic WLL from D7 to D61 of treatment. She was weaned from oxygen on D42 and enteral nutrition on D54, with resolution of vomiting. On D60, all clinical and biological features were dramatically improved (Table 2). Chest CT showed improvement. Echogenicity of the liver normalized. We decided to pursue the treatment after the end of the protocol. She was discharged home on D71. She was admitted for a new assessment at nine months of age, one month after the last WLL. The BAL showed dramatic improvement with total clearance of the extracellular lipoproteinaceous material and a marked decrease in the proportion of vacuolized ORO+macrophages (data not shown). At the last follow-up ten months later, she was asymptomatic. Her weight had reached the mean on the growth curve (Table 2). She was not taking any other treatment apart from methionine and no therapeutic WLL had been performed since D61. Apart from a moderately persistent elevated sedimentation rate, all her laboratory parameters returned to normal (Table 2). Size and echogenicity of the liver normalized. Her chest CT showed discrete postero-basal ground-glass opacities, with no signs of fibrosis (data not shown).

### Patient 2

P2 displayed discrete persistent inflammation at inclusion that resolved at the last follow-up (Table 2). She underwent one WLL which showed only mild lipoproteinaceous material deposition. At the end of the two-month trial, she was starting to eat by herself. We decided to pursue methionine supplementation. After one year of treatment, she showed a significant decrease in her feeding difficulties, along with satisfactory growth (Table 2). Her chest CT, which was already greatly improved after MMF initiation, showed no further changes after methionine supplementation. There was no signs of fibrosis. She did not undergo WLL during the trial nor after. MMF was discontinued.

### Patient 3

At inclusion, P3 displayed a similar presentation as P2 (Table 3), apart from her chest CT that showed a typical crazy-paving aspect (data not shown). Ultrasound showed an enlarged liver. She underwent two therapeutic WLL on D16 and D45. Vomiting stopped on D10. She was weaned from oxygen on D47 and enteral nutrition on D71. On D60, all clinical and biological features were dramatically improved (Table 3). Chest CT showed a clear improvement. The size of the liver decreased. We decided to pursue the treatment after the end of the protocol. She was discharged home on D60. She was admitted for a new assessment at 11 months of age (last follow-up). Her clinical, respiratory, and growth status continued to improve (Table 3). Her chest CT showed new improvement, with no signs of fibrosis (data not shown). The BAL showed partial regression of the extracellular abnormal lipoproteinaceous material and a marked decrease in the number of vacuolized ORO+macrophages (data not shown).

### Patient 4

Before starting methionine, despite repetitive WLL and IV steroid pulses, P4 was severely affected by chronic respiratory insufficiency, requiring continuous NIV, and growth failure, necessitating exclusive parenteral nutrition (Table 3). Chest CT showed a crazy-paving appearance and microcystic lesions suggestive of early-stage fibrosis (data not shown). He was dependent on blood transfusions and albumin perfusions. After starting methionine, he was weaned from NIV on D38, with a progressive decrease in oxygen supply. He was weaned from parenteral nutrition on D87. The last blood transfusion and albumin perfusion were performed on D79 and D56, respectively. A chest CT performed after two months of treatment showed a marked decrease in the density and extension of consolidations, microcystic lesions remained stable (data not shown). The patient has not undergone therapeutic WLL nor received steroids or other treatment since the beginning of methionine supplementation. At the last follow-up, there was a marked catch-up in growth, his anemia and cholestasis had resolved, the albumin plasma levels had improved. He was weaned from oxygen on daytime but still required 0.5 L/min when asleep.

### Comparison to the Historical Controls

We compared the course of P1, 3, and 4 from inclusion (“MO”) to the last follow-up (“M6-M12”) to that of the HC from diagnosis (“MO”) to six months to one year of progression of the disease or at the last evaluation if they died within six months of diagnosis (“M6-M12”). We did not included P2 in this analysis, as all her parameters were normal at inclusion under MMF treatment, except for the nutritional aspect. Data were available for comparison for 20 HC. At diagnosis, 15 patients required supplemental oxygen and 18 enteral nutrition. At the second assessment, 1/15 had been weaned off oxygen and none off enteral nutrition, versus 2/3 patients treated with methionine for both parameters (p=0.056 for oxygen weaning, p=0.014 for enteral nutrition weaning). Among the HC who did not initially required oxygen or enteral nutrition, 2/5 required oxygen and 1/2 enteral nutrition at the second assessment. Differences between values at MO and M6-M12 were statistically significant between HC and treated patients for blood neutrophils (p=0.011), CRP (p=0.039), albumin (p=0.006) and GGT (p=0.038) (data not shown), showing a greater improvement of these parameters for patients treated with methionine than in the HC. We selected chest CT and BAL from two HC at MO and M6-M12. After repetitive WLL for both, associated with systemic steroids for the second, chest CT showed worsening of the lesions in the two patients along with signs of early-stage fibrosis in the second (data not shown). Analyses of their BALF showed the persistence of abundant PAS+macrophages and extracellular PAS+material despite WLL (data not shown).

### Cellular Assays

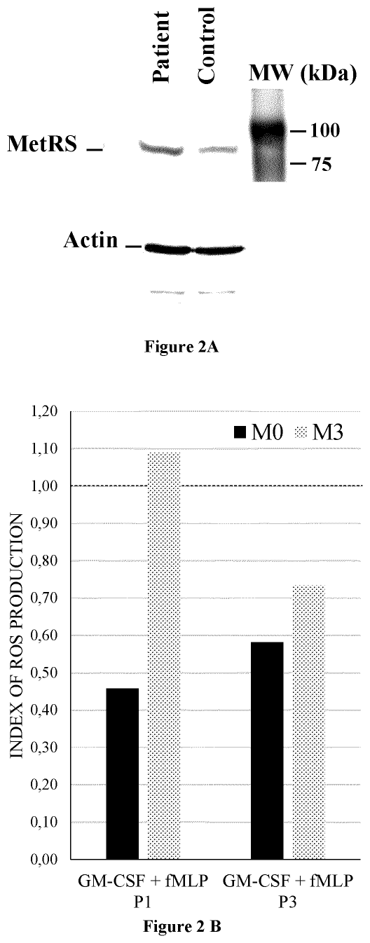

MetRS protein levels in PBMCs of P1 before starting methionine were normal relative to those of a control individual (FIG. 2A).

Before treatment, GM-CSF priming of ROS production by peripheral monocytes of P1 and P3 stimulated by GM-CSF and fMLP was low (stimulation index relative to control of 46% for P1 and 58% for P3). After three months of treatment, the stimulation index relative to control normalized for P1 (109%) and improved for P3 (73%) (FIG. 2B).

### Additionnal Results

Since then, fourteen additional patients were started on methionine, 4 of them since diagnosis (between 3 and 6 months) and 2 before the age of 2 years. For the 4 additional patients treated from diagnosis, methionine supplementation has enabled them to stop whole lung lavages or even to avoid it, to normalize the chest CT scan after 3 months of treatment, to normalize the liver blood tests and inflammatory markers, to catch-up on weight, and to wean off the oxygen and nutritional support initially required. Methionine is their only treatment. For the 2 patients treated between 1 and 2 years of age, methionine allowed to stop whole lung lavages, to “clean” the chest CT scan from the alveolar consolidations and stop the progression of fibrotic lesions, to catch-up on weight and to normalize liver blood tests and inflammatory markers. Eight patients started the treatment from the age of 11 to 24 years old. In 1 patient aged 13 years old, the treatment was started 2 years ago and allowed to improve clinical respiratory status (regression of dry cough and exercise dyspnoea), to improve lung function (gain in forced vital capacity and diffuse capacity of carbon monoxide), and to improve chest CT scan (regression of ground glass opacities, stabilization of the fibrosis). In the 7 seven remaining patients, treatment was started less than one year ago and will be assessed in the next months.

### Conclusion

Methionine supplementation in a patient with PAP related to bi-allelic MARS mutations allowed a dramatic improvement in clinical, biological, imaging, and pathological parameters. The treatment was well tolerated. Additional assays on peripheral monocytes showed an initially altered function that improved under treatment. These results are of particular interest as they are the first to suggest an efficient and well tolerated treatment for this severe disease, with functional data that prove an effect of supplementation at the cellular level. This promising result will fundamentally change the prognosis of this severe and often fatal disease. It also offers perspectives for similar strategies for other ARS deficiencies.

### REFERENCES

Throughout this application, various references describe the state of the art to which this invention pertains. The disclosures of these references are hereby incorporated by reference into the present disclosure.
1. Rosen S H, Castleman B, Liebow A A. Pulmonary alveolar proteinosis. N Engl J Med 1958; 258(23): 1123-42.
2. Holbert J M, Costello P, Li W, Hoffman R M, Rogers R M. CT features of pulmonary alveolar proteinosis. AJR Am J Roentgenol 2001; 176(5): 1287-94.
3. Trapnell B C, Whitsett J A, Nakata K. Pulmonary alveolar proteinosis. N Engl J Med 2003; 349(26):2527-39.
4. Enaud L, Hadchouel A, Coulomb A, et al. Pulmonary alveolar proteinosis in children on La Réunion Island: a new inherited disorder? Orphanet J Rare Dis 2014; 9:85.
5. Hadchouel A, Wieland T, Griese M, et al. Biallelic Mutations of Methionyl-tRNA Synthetase Cause a Specific Type of Pulmonary Alveolar Proteinosis Prevalent on Réunion Island. Am J Hum Genet 2015; 96(5):826-31.
6. Fortowsky G B, Simard D J, Aboelnga M M, Gauld J W. Substrate-Assisted and Enzymatic Pretransfer Editing of Nonstandard Amino Acids by Methionyl-tRNA Synthetase. Biochemistry (Mosc) 2015; 54(37):5757-65.
7. Guo M, Schimmel P. Essential nontranslational functions of tRNA synthetases. Nat Chem Biol 2013; 9(3): 145-53. Yao P, Fox P L. Aminoacyl-tRNA synthetases in medicine and disease. EMBO Mol Med 2013; 5(3):332-43.
9. Comisso M, Hadchouel A, de Blic J, Mirande M. Mutations in MARS identified in a specific type of pulmonary alveolar proteinosis alter methionyl-tRNA synthetase activity. FEBS J 2018; 285(14):2654-61.
10. Trapnell B C, Carey B C, Uchida K, Suzuki T. Pulmonary alveolar proteinosis, a primary immunodeficiency of impaired GM-CSF stimulation of macrophages. Curr Opin Immunol 2009; 21(5):514-21.

11. Rips J, Meyer-Schuman R, Breuer O, et al. MARS variant associated with both recessive interstitial lung and liver disease and dominant Charcot-Marie-Tooth disease. Eur J Med Genet 2018; 61(10):616-20.
12. Chien Y-H, Abdenur J E, Baronio F, et al. Mudd's disease (MAT I/III deficiency): a survey of data for MATIA homozygotes and compound heterozygotes. Orphanet J Rare Dis 2015; 10:99.
13. Garlick P J. Toxicity of methionine in humans. J Nutr 2006; 136 (6 Suppl): 1722S-1725S. 14. Fuchs S A, Schene I F, Kok G, et al. Aminoacyl-tRNA synthetase deficiencies in search of common themes. Genet Med 2019; 21(2):319-30.

1. A method for treating pulmonary alveolar proteinosis related to MARS gene mutations in a subject in need thereof comprising a step of administering to said subject a therapeutically effective amount of methionine and/or a derivative thereof.

2. A method for treating a subject suffering from pulmonary alveolar proteinosis related to MARS gene mutations and who is receiving a supplementation dosage of methionine and/or a derivative thereof, comprising measuring the level of methioninemia in a biological sample obtained from the subject
ii) treating the subject by administering an increased amount of the supplementation dosage when the level of methioninemia is less than a predetermined reference value;
iii) treating the subject by administering the same amount of the supplementation dosage when the level of methioninemia is the same as the predetermined reference value; or
iv) treating the subject by administering a reduced amount of the supplementation dosage when the level of methioninemia is greater than the predetermined reference value.

3. The method according to claim 1, wherein the subject is an adult, child or baby in need thereof.

4. The method according to claim 2, wherein the predetermined reference value is in a range of 45-500 μM.

5. The method according claim 1, wherein the therapeutically effective amount of methionine and/or the derivative thereof is sufficient to reduce and/or control respiratory symptoms, resolve inflammation and cholestasis, increase a rate of catch-up in growth and to free bronchoalveolar lavage fluid of extracellular lipoproteinaceous material.

6. The method according to claim 1, wherein the methionine and/or the derivative thereof is formulated for oral administration.

7. The method according to claim 6, wherein the methionine is formulated as a tablet, a gel capsule, a powders, granules, an oral suspensions or solutions, or a sublingual or buccal administration forms.

8. The method according to claim 1, wherein the therapeutically effective amount of methionine is 0.01 to 500 mg/kg per day.

9. The method according to claim 1, wherein the therapeutically effective amount of methionine and/or the derivative thereof is administered every 6 hours.

10. A pharmaceutical composition comprising methionine and/or a derivative thereof wherein the methionine and/or the derivative thereof is present in the pharmaceutical composition in an amount sufficient to reduce and/or control respiratory symptoms, resolves inflammation and cholestasis, increases a rate of catch-up in growth and free bronchoalveolar lavage fluid of extracellular lipoproteinaceous material when the pharmaceutical composition is administered to a subject suffering from pulmonary alveolar proteinosis related to MARS gene mutations.

11. (canceled)

12. (canceled)

13. A kit for performing the method according to claim 2, wherein said kit comprises (i) means for measuring the level of methioninemia in a biological sample from a subject suffering from pulmonary alveolar proteinosis related to MARS gene mutations and (ii) instructions to adjust the methionine and/or its derivatives supplementation dosage.

14. The method according to claim 2, wherein the subject is an adult, child or baby in need thereof.
